(19) United States
(12) Patent Application Publication
(10) Pub. No.: US 2013/0224298 A1
(43) Pub. Date: Aug. 29, 2013

(54) Two-Phase Preparation And Use Thereof For The Treatment Of Herpes

(71) Applicant: NUTRITEAM GMBH, (US)

(72) Inventor: Ernst-Otto Von Drachenfels, Penzberg (DE)

(73) Assignee: NUTRITEAM GMBH, Penzberg (DE)

(21) Appl. No.: 13/766,973

(22) Filed: Feb. 14, 2013

Related U.S. Application Data
(63) Continuation-in-part of application No. PCT/EP2011/004119, filed on Aug. 16, 2011.

(30) Foreign Application Priority Data
Aug. 16, 2010 (EP) PCT/EP2010/061912

(51) Int. Cl. A61K 45/06; A61K 33/06; A61K 33/04; A61K 36/82; A23L 1/29; A61K 36/87; A61K 31/4188; A61K 31/341; A61K 36/18; A61K 33/30; A61K 36/45

(52) U.S. Cl. CPC A61K 45/06; A61K 33/30; A61K 33/06; A61K 33/04; A61K 36/82; A61K 36/45; A61K 36/87; A61K 31/4188; A61K 31/341; A61K 36/18; A23L 1/296
USPC 424/474; 424/43; 424/643; 424/682; 424/702; 424/729; 424/732; 424/766; 514/388; 514/474; 426/72

(57) ABSTRACT

The invention relates to a novel two-phase preparation for use as a food supplement, dietary substance, and/or medicament. The medical use thereof for helping the immune system fight against viral diseases, especially herpes, is particularly preferred. Said preparation may have two different phases which may be used in a chronologically staggered manner, mutually blocking agents being in different phases and mutually boosting agents being in the same phase.

### RELATED APPLICATIONS AND INCORPORATION BY REFERENCE

This application is a continuation-in-part application of international patent application Serial No. PCT/EP2011/004119 filed 16 Aug. 2011, which published as PCT Publication No. WO 2012/022466 on 23 Feb. 2012, which claims benefit of international patent application Serial No. PCT/EP2010/061912 filed 16 Aug. 2010.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to a novel two-phase preparation. According to the invention, the novel two-phase preparation may be used as a food supplement, dietary substance, and/or medicament.

Preferably, the preparation may be used to support the immune system (e.g., in the case of an acquired immune deficiency). Its medical use for the treatment of viral diseases, especially for the treatment of herpes simplex (caused by the herpes simplex virus), in particular herpes labialis and herpes genitalis, by supporting the immune system is particularly preferred.

### BACKGROUND OF THE INVENTION

Herpes labialis is caused by the herpes simplex virus. Almost 100% of the population is affected by a herpes infection. Usually, the initial infection takes place in early adolescence. Once the herpes virus has entered the body, it will persist in the body for a lifetime. During dormancy, the herpes viruses stay in the trigeminal nerve and are mostly prevented from reactivation by the body's immune system.

However, when the immune system is weakened, a renewed growth phase of the viruses may occur. This mostly occurs in the corners of the mouth, where epithelial cells are infected and are used for the production of new viruses. This occurs at least once a year in approximately 20% of the population, but every 4-8 weeks in a small proportion. This reactivation phase lasts for 11-14 days. The spread of the affected area is usually 3 to 5 mm in size, but may also affect the entire lip. Normally, cold sores do not represent a real health problem. Usually it is a cosmetic problem and rather annoying, but the cracking of the corners of the mouth is also painful. Mostly due to the psychological aspect, cold sores are considered not only a cosmetic problem but often a disease.

Two groups of herpes labialis can be distinguished:
Type 1: This group includes patients, in whom the immune system cannot prevent herpes reactivation because there is a shortage of micronutrients (a shortage thereof in the body). These include competitive athletes and high-risk groups, for which the shortage occurs nutritionally.
Type 2: The so-called disgust-induced herpes group does not suffer from recurrent herpes reactivations because of a shortage but because of other causes (e.g., psychological causes, such as disgust).

Many combined preparations are commercially available which contain vitamins, minerals, trace elements, and plant constituents and which improve and support health, performance, and regeneration. However, the available preparations are often not balanced in their composition, which means that the individual components of the preparations are not mutually supportive but competitive. Thus, the positive effects of each ingredient element do not occur, or only occur in reduced form. In such a case, one often speaks of antagonists or inhibitors.

Citation or identification of any document in this application is not an admission that such document is available as prior art to the present invention.

### SUMMARY OF THE INVENTION

An object of the present invention is to provide a preparation which may comprise vitamins and plant constituents, and optionally minerals and/or trace elements, and which eliminates or reduces the mutual interference of the individual components, on the one hand, and favors the mutual support to improve the intake and/or effect of the individual ingredient element in the human body, on the other hand. Preferably, the active ingredients, which are administered simultaneously, show a synergistic effect, i.e. the desired effect of the individual active ingredient is enhanced by co-administration. In a specific embodiment, the preparation according to the invention may be used as a food supplement and/or a dietary substance.

A further object of the present invention is to provide a preparation to improve and support health and regeneration, and/or to support the immune system. Preferably, support of the immune system serves the treatment of viral infections (i.e., of viral diseases). The immune system is supported in the event of a viral disease. In particular, an object of the present invention is to provide a preparation for the treatment of herpes by supporting the immune system, such as for the treatment of herpes simplex, in particular herpes labialis (cold sores) and herpes genitalis.

The problem underlying the present invention is solved by the provision of a preparation which has two different phases (unit dose forms), which are used in a chronologically staggered manner (i.e., taken or administered), wherein mutually blocking agents are in different phases and mutually booting agents are in the same phase.

In a first embodiment, the present invention is directed to a two-phase preparation for use in a chronologically staggered manner which may comprise
(a) a phase (A) which may comprise, in addition to water-soluble vitamins and/or the corresponding pro-vitamins, plant constituents and optionally minerals and/or trace elements, in a first unit dose form
(b) a phase (B) which may comprise, in addition to fat-soluble vitamins and/or the corresponding pro-vitamins, plant constituents and optionally minerals and/or trace elements, in a second unit dose form,
   characterized in that phase (A) preferably contains grape seed extract and/or cranberry juice powder as plant constituents and that phase (B) preferably contains green tea extract and/or pomegranate extract as plant constituents. Preferably, phase (A) does not contain any green tea extract.

In a second embodiment, the present invention is directed to a kit which may comprise
(a) a phase (A) which may comprise, in addition to water-soluble vitamins and/or the corresponding pro-vitamins, plant constituents and optionally minerals and/or trace elements, in a first unit dose form
(b) a phase (B) which may comprise, in addition to fat-soluble vitamins and/or the corresponding pro-vitamins, plant constituents and optionally minerals and/or trace elements, in a second unit dose form,
   characterized in that phase (A) preferably contains grape seed extract and/or cranberry juice powder as plant constituents and that phase (B) preferably contains green tea extract and/or pomegranate extract as plant constituents.

The unit dose forms of the phases (A) and/or (B) are oral dosage forms, typically selected from the group consisting of capsules, tablets, coated tablets, pills, granules, effervescent tablets, powders, drinkable solutions and suspensions. Preferred dosage forms are capsules.

It is important for the present invention that the two phases (A) and (B) are applied in a chronologically staggered manner, meaning that they are not to be taken simultaneously. The time interval is preferably at least 2 hours and preferably not more than 28 hours, and may be, for example, 2, 5, 8, 12, 24, or 28 hours. A time interval of more than 28 hours is only appropriate in exceptional cases. Taking the phases (A) and (B) alternating at a time interval of 24 hours has been found to be particularly positive. Thereafter, it is particularly advantageous when
(a) the phase (A) is applied to meals on days X+2N, where “N” represents the non-negative integers and
(b) the phase (B) is applied to meals on days X+(2N+1), where “N” represents the non-negative integers.

In one embodiment, the phases (A) and (B) are administered at the same time of day, preferably with a meal, for example, for breakfast or dinner, but on alternate days.

In one embodiment, the two-phase preparation according to the invention is for use as a medicament, particularly for use in the treatment and/or prevention of herpes simplex, and in particular herpes labialis. Here, in one aspect of the invention, the administration (dosage) is applied in a chronologically staggered manner as described above, especially on alternate days at the same time of day.

The two-phase preparation according to the invention may be used to protect cells from free radicals (e.g., caused by oxidative stress) in vivo and in vitro.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of and “consists essentially of have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE FIGURE

The following detailed description, given by way of example, but not intended to limit the invention solely to the specific embodiments described, may best be understood in conjunction with the accompanying drawings.

FIG. 1 shows the effect of the two-phase preparation according to the invention on a cell culture under oxidative stress. See Example 4. Left column: without oxidative stress, without the two-phase preparation according to the invention (control); middle column: with oxidative stress, but without the two-phase preparation according to the invention; right column: with oxidative stress, with the two-phase preparation according to the invention.

### DETAILED DESCRIPTION OF THE INVENTION

The present invention teaches that the active ingredients contained in grape seed extract are advantageously used in combination with water-soluble vitamins, pro-vitamins and the optionally present minerals and/or trace elements (referred to in the following as phase (A)) and that the active ingredients contained in green tea extract are advantageously used in combination with fat-soluble vitamins, pro-vitamins and the optionally present minerals and/or trace elements (referred to in the following as phase (B)). Advantageously, the phases (A) and (B) are applied in a chronologically staggered manner.

Grape seed extract contains active ingredients similar to the active ingredients contained in cranberry juice powder. Accordingly, in one embodiment grape seed extract may be replaced by cranberry juice powder. The water-soluble vitamins, applied together with the grape seed extract and cranberry juice powder, are particularly preferred.

Green tea extract contains active ingredients similar to the active ingredients contained in pomegranate extract. Accordingly, in one embodiment green tea extract may be replaced by pomegranate extract. The fat-soluble vitamins, applied together with the green tea extract and pomegranate extract, are particularly preferred.

In the following, the phases (A) and (B) are described in more detail.

### Phase (A)

According to the present invention, phase (A) may comprise, in addition to water-soluble vitamins (and/or corresponding pro-vitamins), preferably grape seed extract and/or cranberry juice powder. Preferably, phase (A) does not contain any vitamin C, biotin, fat-soluble vitamins and/or green tea extract.

It is believed that the active ingredients (such as procyanidine and proanthocyanidins) contained in grape seed extract and cranberry juice powder, are not antagonists or inhibitors, respectively, and that they may even act synergistically in phase (A). Thus, phase (A) advantageously may comprise both grape seed extract and cranberry juice powder.

If phase (A) contains grape seed extract, phase (A) advantageously may comprise 40 mg-160 mg of grape seed extract, preferably 60 mg-120 mg of grape seed extract, and most preferably 75 mg-85 mg of grape seed extract.

If phase (A) contains cranberry juice powder, phase (A) advantageously may comprise

20 mg-80 mg of cranberry juice powder, preferably 30 mg-60 mg of cranberry juice powder, and most preferably 35 mg-45 mg of cranberry juice powder.

The preferred water-soluble vitamins of phase (A) include D-pantothenic acid (vitamin B₅, preferably as calcium D-pantothenate), niacin (vitamin B₃, preferably as nicotinamide), vitamin B₆ (preferably as pyridoxine HCl), riboflavin (vitamin B₂), vitamin B₁ (preferably as thiamine mononitrate), vitamin B₁₂ (preferably cyanocobalamin), and folic acid (vitamin B₉). In one embodiment, phase (A) may comprise, in addition to grape seed extract and/or cranberry juice powder, all of the above-mentioned preferred vitamins (i.e., D-pantothenic acid (vitamin B₅, preferably as calcium D-pantothenate), niacin (vitamin B₃, preferably as nicotinamide), vitamin B₆ (preferably as pyridoxine HCl), riboflavin (vitamin B₂), vitamin B₁ (preferably as thiamine mononitrate), vitamin B₁₂ (preferably cyanocobalamin), and folic acid.

The preferred water-soluble vitamins (regardless of whether all or only some of these vitamins are contained in phase (A)) are advantageously used in the following amounts:

8 mg-28 mg of D-pantothenic acid, preferably 13 mg-23 mg of D-pantothenic acid

6 mg-26 mg of niacin, preferably 12 mg-20 mg of niacin

1 mg-7 mg of vitamin B₆, preferably 3.5 mg-4.9 mg of vitamin B₆

1 mg-7 mg of riboflavin, preferably 3 mg-5 mg of riboflavin

1 mg-7 mg of vitamin B₁, preferably 2.5 mg-4.1 mg vitamin B₁

3 μg-12 μg of vitamin B₁₂, preferably 6 μg-9 μg of vitamin B₁₂,

0.1-1 mg of folic acid, preferably 0.2-0.6 mg of folic acid.

As free D-pantothenic acid is chemically unstable, the corresponding amount of calcium D-pantothenate is preferably used. Vitamin B₆ is usually used as pyridoxine HCl. Vitamin B₁ is usually used as thiamine mononitrate. Vitamin B₁₂ is preferably used as 1% inulin powder preparation.

In another embodiment, phase (A) additionally may comprise selenium and/or magnesium. Selenium is preferably used as sodium selenite, and in the form of a 1% maltodextrin powder preparation. Magnesium is preferably used as magnesium citrate.

If phase (A) contains selenium, phase (A) advantageously may comprise 20 μg 90 μg of selenium, preferably 45 μg-65 μg of selenium, preferably in the form of sodium selenite.

If phase (A) contains magnesium, phase (A) advantageously may comprise 30 mg-90 mg of magnesium, more preferably 50 mg-70 mg of magnesium, preferably in the form of magnesium citrate.

The unit dose forms (dosage forms) of phase (A) are typically oral dosage forms, such as capsules, tablets, coated tablets, pills, granules, effervescent tablets, powders, drinkable solutions or suspensions. Preferably, phase (A) is administered as a capsule. In one aspect of the invention, phase (A) is a blue-colored capsule.

Accordingly, phase (A) may further comprise pharmaceutical excipients, such as magnesium stearate (typically 5 mg to 15 mg), silicon dioxide (typically 1 mg to 5 mg), titanium dioxide (typically 0.1 mg to 3 mg), gelatin (typically 50 mg to 150 mg), and/or dyes (such as, for example, Indigo Carmine-Blue 2).

In one embodiment, phase (A) may comprise or contains:
(i) 30 mg-90 mg of magnesium, preferably in the form of magnesium citrate
(ii) 40 mg-160 mg of grape seed extract
(iii) 20 mg-80 mg of cranberry juice powder
(iv) 8 mg-28 mg of D-pantothenic acid, preferably in the form of calcium D-pantothenate
(v) 6 mg-26 mg of niacin, preferably in the form of nicotinamide
(vi) 20 μg-90 μg of selenium, preferably in the form of a maltodextrin powder preparation which may comprise 1% sodium selenite
(vii) 1 mg-7 mg of vitamin B₆, preferably in the form of pyridoxine HCl
(viii) 1 mg-7 mg of riboflavin
(ix) 1 mg-7 mg of vitamin B₁, preferably in the form of thiamine mononitrate
(x) 3-12 μg of vitamin B₁₂, preferably as inulin powder preparation which may comprise 1% cyanocobalamin
(xi) 0.1-1 mg of folic acid
(xii) excipients, such as magnesium stearate, silicon dioxide, titanium dioxide, gelatin, and/or dyes (such as, for example, Indigo Carmine-Blue 2).

### Phase (B)

According to the present invention, phase (B) may comprise, in addition to fat-soluble vitamins (and/or corresponding pro-vitamins), preferably green tea extract and/or pomegranate extract. Preferably, phase (B) does not comprise, in addition to fat-soluble vitamins (and/or corresponding pro-vitamins), any water-soluble vitamins (and/or corresponding pro-vitamins), apart from biotin and vitamin C. In one embodiment, phase (B) does not contain any grape seed extract and/or grape skin extract.

It is believed that the active ingredients (such as, for example, catechine) contained in green tea extract and pomegranate extract are not antagonists or inhibitors, respectively, and that they may even act synergistically in phase (B). Thus, phase (B) advantageously may comprise both green tea extract and pomegranate extract.

If phase (B) contains green tea extract, phase (B) advantageously may comprise
5 mg-160 mg of green tea extract, preferably 40 mg-120 mg of green tea extract, and most preferably 70 mg-90 mg of green tea extract.

If phase (B) contains pomegranate extract, phase (B) advantageously may comprise
5 mg-80 mg of pomegranate extract, preferably 20 mg-60 mg of pomegranate extract, and most preferably 30 mg-50 mg of pomegranate extract.

The preferred fat-soluble vitamins of phase (B) include vitamin E (tocopherol), vitamin D (preferably vitamin D₃), and beta-carotene. In one embodiment, phase (B) may comprise, in addition to green tea extract and/or pomegranate extract, all of the above-mentioned preferred vitamins (i.e., vitamin E, vitamin D, and beta-carotene).

The preferred fat-soluble vitamins (regardless of whether all or only some of these vitamins are contained in phase (B)) are advantageously used in the following amounts:
2 mg-6 mg of beta-carotene, preferably 4 mg-5.6 mg of beta-carotene, preferably as a 10% preparation
5 mg-35 mg of vitamin E, preferably 11 mg-25 mg of vitamin E, preferably in the form of dl-alpha-tocopheryl acetate
0.5 μg-10 μg of vitamin D, preferably 3 μg-7 μg of vitamin D, preferably as cholecalciferol (vitamin D₃) with preferably 100.000 IU/g.

Further fat-soluble vitamins, which may be contained in phase (B), include among others retinol (vitamin A) and/or vitamin K and their corresponding pro-vitamins.

In a further embodiment, phase (B) additionally may comprise vitamin C, biotin, and/or zinc as a trace element, preferably in the form of zinc salt, such as zinc gluconate.

If phase (B) contains vitamin C, phase (B) advantageously may comprise 120 mg-480 mg of vitamin C, preferably 180 mg-300 mg of vitamin C, and most preferably 220 mg-260 mg of vitamin C.

If phase (B) contains zinc, phase (B) advantageously may comprise 1 mg-20 mg of zinc, and more preferably 2 mg-10 mg of zinc, preferably in the form of zinc gluconate.

If phase (B) contains biotin, phase (B) advantageously may comprise 20 μg-200 μg of biotin, and more preferably 50 μg-150 μg biotin, preferably as 1% by weight D-biotin dry glucose preparation.

The unit dose forms (dosage forms) of phase (B) are typically oral dosage forms, such as capsules, tablets, coated tablets, pills, granules, effervescent tablets, powders, drinkable solutions or suspensions. Preferably, phase (B) is administered as a capsule. In one aspect of the invention, phase (B) is a red- or an orange-colored capsule.

Accordingly, phase (B) may further comprise pharmaceutical excipients, such as magnesium stearate (typically 5 mg to 15 mg), silicon dioxide (typically 1 mg to 5 mg), titanium dioxide (typically 0.1 mg to 3 mg), gelatin (typically 50 mg to 150 mg) and/or dyes (such as, for example, iron oxide red).

In one embodiment, phase (B) may comprise or contains:
(i) 120 mg-480 mg of vitamin C
(ii) 5 mg-160 mg of green tea extract
(iii) 2 mg-6 mg of beta-carotene, preferably as 10% preparation
(iv) 1 mg-20 mg of zinc, preferably as zinc gluconate
(v) 5 mg-80 mg of pomegranate extract
(vi) 5 mg-35 mg vitamin E, preferably as dl-alpha-tocopheryl acetate
(vii) 20 μg-200 μg biotin, preferably as 1% by weight D-biotin dry glucose preparation
(viii) 0.5 μg-10 μg of vitamin D, preferably as cholecalciferol (vitamin D₃) with preferably 100.000 IU/g
(ix) excipients, such as for example magnesium stearate, silicon dioxide, titanium dioxide, gelatin, and/or dyes (such as, for example, iron oxide red).

In a more preferred embodiment, phase (B) may comprise or contains:
(i) 120 mg-480 mg of vitamin C
(ii) 40 mg-160 mg of green tea extract
(iii) 4 mg-5.6 mg beta-carotene, preferably as 10% preparation
(iv) 2 mg-10 mg zinc, preferably as zinc gluconate
(v) 20 mg-60 mg pomegranate extract
(vi) 11 mg-25 mg of vitamin E, preferably as dl-alpha-tocopheryl acetate
(vii) 50 μg-150 μg of biotin, preferably as 1% by weight D-biotin dry glucose preparation
(viii) 3 μg-7 μg of vitamin D, preferably as cholecalciferol (vitamin D₃) with preferably 100.000 IU/g
(ix) excipients, such as for example magnesium stearate, silicon dioxide, titanium dioxide, gelatin, and/or dyes (such as, for example, iron oxide red).

Embodiments as a capsule, which are the subject of the present invention, are described in Example 1A and Example 1B.

### Interaction Between Phase (A) and Phase (B)

According to the invention, the phases (A) and (B) are administered in a chronologically staggered manner, meaning that they are not to be taken simultaneously. Thus, in one embodiment the present invention pertains to a kit which may comprise
(a) a phase (A) which may comprise, in addition to water-soluble vitamins and/or the corresponding pro-vitamins, plant constituents and optionally minerals and/or trace elements, in a first unit dose form
(b) a phase (B) which may comprise, in addition to fat-soluble vitamins and/or the corresponding pro-vitamins, plant constituents and optionally minerals and/or trace elements, in a second unit dose form,
   characterized in that phase (A) preferably contains grape seed extract and/or cranberry juice powder as plant constituents and that phase (B) preferably contains green tea extract and/or pomegranate extract as plant constituents, wherein phases (A) and/or (B) are oral dosage forms, typically selected from the group consisting of capsules, tablets, coated tablets, pills, granules, effervescent tablets, powders, drinkable solutions and suspensions. Preferred are phases (A) and (B) as capsules.

The two-phase preparation according to the invention may be used to protect living cells from free radicals, such as they may result from oxidative stress. Such an application is possible in vivo (for example, as a food supplement) and in vitro (for example, by addition to a cell culture).

The two-phase preparation according to the invention may support the immune system (e.g., in the case of a herpes simplex infection).

Moreover, the present invention relates to the use of the two-phase preparation according to the invention, which may comprise phases (A) and (B), as a food supplement or dietary substance. Likewise, the present invention pertains to the use of the kit which may comprise phases (A) and (B) according to the invention as a food supplement or dietary substance.

In a preferred embodiment, the present invention pertains to the use of the two-phase preparation according to the invention, which may comprise phases (A) and (B), as a medicament. Likewise, the present invention pertains preferably to the use of the kit according to the invention, which may comprise phases (A) and (B), as a medicament.

In a particularly preferred embodiment, the present invention pertains to the use of the two-phase preparation according to the invention, which may comprise phases (A) and (B), for the treatment and/or prevention of herpes, such as herpes simplex, and in particular for the treatment of herpes labialis and/or herpes genitalis, especially herpes labialis. Also, the present invention relates preferably to the use of the kit according to the invention, which may comprise phases (A) and (B), for the treatment and/or prevention of herpes, such as herpes simplex, and in particular for the treatment of herpes labialis and/or herpes genitalis, especially herpes labialis.

In particular, the present invention relates to the use of the two-phase preparation according to the invention, which may comprise phases (A) and (B), for the treatment and/or prevention of recurring outbreaks of herpes, such as herpes simplex, and in particular for the treatment of herpes labialis and/or herpes genitalis, especially herpes labialis. Also, the present invention relates preferably to the use of the kit according to the invention, which may comprise phases (A) and (B), for the treatment and/or prevention of recurring outbreaks of herpes, such as herpes simplex, and in particular for the treatment of herpes labialis and/or herpes genitalis, especially herpes labialis.

The treatment and/or prevention of herpes according to the invention may be applied to all patients infected with herpes, in particular herpes simplex (i.e., patients from both groups: type 1 and type 2). Preferably, for type 1 a reduction in the number of recurring outbreaks (herpes reactivations), up to a complete absence of recurrences is achieved. Preferably, for type 2 a reduction in the severity of recurring outbreaks is achieved, and in particular a reduction in the size of the lesions and/or a reduction in the process. However, these treatment results may also occur in the other respective group.

Without being bound by theory, the inventors believe that the use according to the invention as a medicament is based on the fact that by administering the two-phase preparation according to the invention the immune system of the patient is supported and that the effect against herpes is at least in part, but possibly also completely, based on this support of the immune system.

A method for the treatment and/or prevention of said diseases by the administration of the two-phase preparation according to the invention to a patient is also one object of the present invention. The administration is performed in a chronologically staggered manner.

A further object of the present invention is the use of the two-phase preparation according to the invention for preparing a medicament for use according to the invention as a medicament as described above.

It is understood that even with the use of a two-phase preparation according to the invention as a food supplement, the effect against herpes might occur. The use of the two-phase preparation according to the invention for cosmetic purposes (i.e., to reduce or avoid the cosmetically undesirable lesions associated with herpes) is therefore also an object of the present invention.

It is important for all uses according to the invention that the two phases (A) and (B) be used in a chronologically staggered manner, meaning that they are not to be taken simultaneously. The time interval is preferably at least 2 hours and preferably not more than 28 hours, and may be for example 2, 5, 8, 12, 24 or 28 hours. A time interval of more than 28 hours is only appropriate in exceptional cases. Taking phases (A) and (B) alternating at a time interval of 24 hours has been found to be particularly positive. Thereafter, it is particularly advantageous when

(a) the phase (A) is applied to meals on days X+2N,

(b) the phase (B) is applied to meals on days X+(2N+1).

“N” represents the non-negative integers (i.e., 0, 1, 2, 3, 4 etc.) and “X” represents any day, in particular “X” is the day on which the administration of the preparation or of the kit begins.

In one preferred embodiment, phases (A) and (B) are taken for breakfast or dinner, but on alternate days.

Regardless of the time interval between administrations, therapy and treatment, etc. are preferably started with phase (A).

The term “vitamins” encompasses all organic compounds which are not used by the human body as an energy source (but rather for other vital functions) that cannot be synthesized by the metabolism (with few exceptions). Therefore, vitamins must be taken in from food. A person skilled in the art understands that vitamins may be taken in in various forms (e.g., in the form of pharmaceutically acceptable salts). For example, vitamin B₆ may be taken in as pyridoxine HCl, and vitamin B₁ as thiamine mononitrate.

“Pro-vitamins” are precursors of vitamins that have yet to be converted into the corresponding vitamins. For example, the pro-vitamin D₃ (7-dehydrocholesterol) is converted to vitamin D₃ (cholecalciferol) in the skin by UV radiation such as sunlight. n-Carotene in turn represents the pro-vitamin of retinol (vitamin A).

In connection with the present invention, “plant constituent” refers to whole plants, plant parts, active ingredients isolated from plants, and substances obtained by chemical synthesis, which are chemically identical to active ingredients isolated from plants. Preferably, in connection with the present invention “plant constituent” refers to whole plants or plant parts (e.g., flowers, leaves, seeds, bark, roots), raw or crushed drugs called precursors, or juices, tinctures, extracts, teas, which may also be further processed into powder or gels, etc., or may be employed as preparations, triturations, or applied to a matrix.

In connection with the present invention, the term “active ingredient” refers to a substance in an organism (typically in a mammal, such as a human) that causes a specific effect and is typically administered in a low dose. Exemplary active ingredients are polyphenols, caffeine, ellagic acid, proanthocyanidine and procyanidins.

In connection with the present invention, the term “extract” refers to a drug extract (also called extract of medicinal drugs or drug extract) which is obtained by means of extraction from a raw material. Possible raw materials are, for example, green tea, pomegranate and grape seeds. In case the extract is in liquid form, it is called (depending on the form of preparation of tinctures or fluid extracts) liquid extract(s). By evaporation of the extraction agent and/or other liquids, the extract may be viscous or solid (for example, in form of a powder). The term “extract” is used regardless of the concentration of substances present therein.

In connection with the present invention, the term “prepare” refers to a mechanical procedure in the field of pharmacy/pharmaceutical technology, which is typically used for better handling and/or dosage of low-dose drugs in dosage forms. When preparing a “preparation”, an active ingredient is mixed one or more times with a suitable base (preferably inulin, maltodextrin, lactose, etc.) to achieve homogeneity (i.e., uniform distribution of the active ingredient in the base). According to the invention, preparations in powder form (i.e., “powder preparations”) are preferred.

In connection with the present invention, a “dietary substance” (dietetic) differs from common foodstuffs by its special composition and/or through a special manufacturing process. In particular, products are included that are intended for the dietary treatment of patients with a specific medical condition or for a nutritional formulation customized to treat a particular disease or disorder.

In connection with the present invention, the term “kit” refers to a set of parts. The kit according to the invention may comprise, in addition to phases (A) and (B), further components such as a leaflet and other food substances and/or medicaments.

“Green tea” refers to tea leaves, which are not fermented like black tea. For this reason, almost all active ingredients contained in the fresh leaf remain preserved. “Green tea extract” refers to an extract of green tea as a raw material. Preferred is a green tea extract containing at least 1% by weight (preferably at least 50% by weight) polyphenols and/or at least 1% by weight (preferably at least 7% by weight) caffeine, preferably at least 1% by weight polyphenols as well as at least 1% by weight of caffeine. Further preferred is “green tea extract” from the species Camellia sinensis L. Particularly preferred is “green tea extract” from the species Camellia sinensis L. in powder form, which has the indicated polyphenols and caffeine amounts.

The pomegranate is a plant species, specifically a berry bush species, which is usually attributed to the family of loosestrife plants (Lythracea). Their red fruit is eaten as a fruit. The “pomegranate extract” according to the invention is typically an extract (typically in a ratio of 15:1) of this fruit in powder form. Preferably, it is an extract of the starting material Punica granatum L. The content of ellagic acid (HPLC) in the extract is preferably at least 1% by weight, more preferably at least 20% by weight, more preferably at least 30% by weight, and most preferably at least 40% by weight.

Cranberry, also known as Vaccinium oxycoccos, is a berry bush species of blueberries (Vaccinium) from the family Ericaceae. The fruits of American cranberries are widely cultivated and marketed in the U.S. and elsewhere. Particularly suitable is the American species Vaccinium macrocarpum, which is converted into cranberry juice concentrate. The “cranberry juice powder” according to the invention is a powder made from the juice of cranberry fruits. Preferred is the juice of the American species Vaccinium macrocarpum. Typically for the preparation of the powder, the pressed cranberry juice is applied as a concentrate to a matrix of maltodextrin, preferably in a ratio of 60% maltodextrin to 40% cranberry juice concentrate. The content of oligomeric proanthocyanidins (OPC, also referred to as “condensed tannins”) in the cranberry juice powder is preferably at least 1% by weight, more preferably at least 10% by weight, more preferably at least 30% by weight, and most preferably at least 70% by weight.

“Grape seed extract” according to the present invention is an extract from the seeds of grapes, preferably from the wine fruit Vitis vinifera L. In particular, it is an extract (typically in a ratio of 50:1) in powder form. The content of polyphenols (FC) in the extract is preferably at least 1% by weight, more preferably at least 10% by weight, more preferably at least 30% by weight, and most preferably at least 95% by weight.

It is possible (though not always preferred) to replace the above-mentioned active ingredients of plant origin by extracts, isolates, or even the synthetically produced main chemical components and/or to supplement or enrich them.

The term “inulin preparation” refers to a dosage form, in the preparation of which the starting material is dispensed once or several times with inulin by a suitable method. Preferred is a preparation based on inulin in powder form (i.e., an inulin powder preparation). When specifying “1% inulin preparation”, etc., this refers to the percentage by weight.

The term “maltodextrin preparation” refers to a dosage form, in the preparation of which the starting material is dispensed once or several times with maltodextrin by a suitable method. Preferred is a preparation based on maltodextrin in powder form (i.e., a maltodextrin powder preparation). When specifying “1% maltodextrin preparation”, etc., this refers to the percentage by weight.

When specifying “beta-carotene 10% preparation” or “β-carotene 10% preparation”, etc., the percentages are by weight. Preferred is a 10% powder preparation. Therein vegetable fat, fish gelatin, glucose syrup, vitamin E, ascorbyl palmitate, and tricalcium phosphate are used as a matrix. The amount of beta-carotene in food supplements is often expressed in “retinol equivalent”. Thereby, 1 mg of retinol corresponds to 6 mg of beta-carotene.

When specifying “D-biotin 1% preparation”, etc., the percentages are by weight. Preferred is a 1% powder preparation based on maltodextrin.

In the term “100.000 IU/g”, etc., “IU” refers to International Unit. For vitamin D₃ it is as follows: 40 IU of vitamin D₃ (1 mg of vitamin D₃ 100.000 IU/g=0.025 μg of vitamin D₃)=1 μg of vitamin D₃, 1 IU of vitamin D₃ [symbol] 0.025 μg of vitamin D₃ [symbol] 65.0 pmol of vitamin D₃. The concentration term “IU/g” refers to the amount of vitamin D₃ per gram of a vitamin D₃-containing mixture.

In the present invention, the term “folic acid” refers preferably to pteroylmonoglutamic acid, but may also refer to any folates.

In the present invention, the term “magnesium” refers to magnesium ions (typically Mg²⁺), the term “selenium” to selenium ions (typically Se⁴⁺), and the term “zinc” to zinc ions (typically to Zn²⁺).

In the present invention, the term “excipient” refers especially to a pharmaceutical excipient, which is used in addition to the active ingredients when preparing phases (A) and (B). Pharmaceutical excipients may have different functions, such as shaping (excipients that carry the active ingredient and give the medicament its form), manufacturability (excipients that enable or improve certain production steps in medicament production) and/or controlling the release of the active ingredients (excipients that cause the active ingredients to be released quickly, slowly, delayed, or otherwise modified), stability enhancement (excipients that provide sufficient shelf life of a medicament). Other properties such as physiological tolerance, color, smell and taste may also be set by pharmaceutical excipients.

Although the present invention and its advantages have been described in detail, it should be understood that various changes, substitutions and alterations can be made herein without departing from the spirit and scope of the invention as defined in the appended claims.

The following examples describe the invention in detail, however, they shall not be considered as limiting the invention.

### EXAMPLES

### Example 1A

### Two-Phase System

The following two-phase system has been proven to be particularly suitable:

Blue Capsule (Phase (A))

| | | |
|---|---|---|
| Magnesium citrate | 350-450 | mg |
| Grape seed extract | 70-90 | mg |
| Cranberry juice powder | 30-50 | mg |
| Calcium D-pantothenate (vitamin B₅) | 15-25 | mg |
| Nicotinamide (vitamin B₃) | 12-20 | mg |
| Sodium selenite 1% maltodextrin preparation | 8-16 | mg |
| Pyridoxine HCl (vitamin B₆) | 3-8 | mg |
| Riboflavin | 2-6 | mg |
| Thiamine mononitrate (vitamin B₁) | 2-6 | mg |
| Cyanocobalamin (vitamin B₁₂), 1% inulin | 1-2.5 | mg |
| preparation | | |
| Folic acid | 0.2-0.6 | mg |
| Magnesium stearate | 7-11 | mg |
| Silicon dioxide | 2-4 | mg |
| Titanium dioxide | 1-3 | mg |
| Indigo Carmin-Blue 2 | 0.04-0.14 | mg |
| Gelatin | 80-110 | mg |

Orange Capsule (Phase (B))

| | | |
|---|---|---|
| Vitamin C (L-ascorbic acid) | 220-260 | mg |
| Green tea extract | 40-80 | mg |
| β-Carotene, 10% preparation | 45-55 | mg |
| Zinc gluconate | 40-55 | mg |
| Pomegranate extract | 30-50 | mg |
| dl-α-Tocopheryl acetate, (vitamin E) | 30-40 | mg |
| D-Biotin, 1% preparation | 5-15 | mg |
| Cholecalciferol (vitamin D₃), 100.000 IU/g | 1-3 | mg |
| Magnesium stearate | 5-10 | mg |
| Silicon dioxide | 2-4 | mg |
| Iron oxide red | 1-2 | mg |
| Titanium dioxide | 0.3-0.7 | mg |
| Gelatin | 80-110 | mg |

### Example 1B

### Two-Phase System

The following two-phase system, which was used in Examples 2 to 4, has also been proven to be particularly suitable:

Blue Capsule (Phase (A)):

| | | |
|---|---|---|
| Niacin (nicotinamide) | 16 | mg |
| Vitamin B₁ (thiamine) | 3.3 | mg |
| Vitamin B₂ (riboflavin) | 4.2 | mg |
| Vitamin B₅ (pantothenic acid) | 18 | mg |
| Vitamin B₆ (pyridoxine) | 4.2 | mg |
| Vitamin B₉ (folic acid) | 0.4 | mg |
| Vitamin B₁₂ | 7.5 | μg |
| Magnesium | 60 | mg |
| Selenite | 55 | μg |
| Grape seed extract | 80 | mg |
| Cranberry juice powder | 40 | mg |

Orange Capsule (Phase (B)):

| Vitamin C | 240 | mg |
|---|---|---|
| Vitamin E | 18 | mg |
| Biotin | 100 | μg |
| Vitamin A (beta-carotene) | 800 μg of | |
| | retinol equivalent, | |
| | hence 4.8 mg of | |
| | beta-carotene | |
| Vitamin D | 5 | μg |
| Zinc | 6 | mg |
| Green tea extract | 80 | mg |
| Pomegranate extract | 40 | mg |

### Example 2

### Effectiveness in Competitive Athletes

During the development of the two-phase preparation according to the invention many competitive athletes were involved, including Olympic athletes. Competitive athletes are known to suffer from the often called “Open Window” effect after difficult competitions or hard training sessions. Due to the high performances, the immune system is weakened, which consequently leads to different bagatelle infections, such as flu or herpes labialis. During the development phase of the two-phase preparation according to the invention, competitive athletes repeatedly reported that by taking the two-phase preparation of Example 1B in 24 hours alternating the blue and orange capsule, herpes labialis did not occur, but after stopping the intake of the two-phase preparation according to the invention it re-occurred.

### Example 3

### Clinical Trials

In a clinical trial on the effectiveness in preventing herpes labialis, the two-phase preparation according to the invention was tested on 30 subjects as described in Example 1B, who were all non-competitive athletes.

Summary of the results for the two groups of herpes labialis types, as described in “Background of the invention” section:
Type 1: The immune system cannot prevent herpes reactivation because there is a shortage (e.g., competitive athletes). In this group, the two-phase preparation according to the invention could strengthen the immune competence so that the herpes reactivation failed. This group included 40% of the subjects.
Type 2: Recurrent herpes reactivations were observed. The processes, however, were significantly shorter than without the two-phase preparation according to the invention. They were shortened from 10-14 days to about 3 days. Also, the severity of the lesions was significantly lower. Personal well-being on a scale of 1-10 increased from 6-8 to 1-2. This group included 60% of the subjects.

### Included Subjects

Subjects who suffered periodically from herpes labialis (i.e. from a reactivation phase every four weeks to three months) were included. These subjects were observed for six months.

Here, the results of 30 subjects are presented, all of which originate from one test center and were always overseen by the same doctor:
30 subjects, aged 20 to 71 years. The median was 26 years of age. 80% were female, 20% male.

As the cause of herpes reactivation, stress was specified by all subjects, in addition to nauseation 63% and strong sunlight radiation exposure 17%.

The previously performed countermeasures during the reactivation phase were mostly administration of acyclovir, but also applying toothpaste or Penaten cream.

### Procedure

The patients were instructed to alternately consume the two-phase preparation according to the invention, one orange and one blue capsule in alternation. On one day an orange capsule should have been taken, and on the next day a blue capsule. Furthermore, no other countermeasures should have been taken.

### Results

60% of subjects suffered from recurring outbreaks of herpes labialis in the observed period of six months. It has been suggested that this is the disgust-induced herpes type (type 2, see above). In contrast to competitive athletes, the reduction of immune competence is not based on a pure shortage of micronutrients, but could be based on psychological factors.

This group (“disgust-induced herpes group”) stated in agreement that the severity and duration of the disease had improved significantly compared to previous experiences. The duration of herpes reactivation was specified with 10-14 days without the two-phase preparation according to the present invention. With the two-phase preparation according to the present invention, the outbreak was reduced to about three days. Here, the size of the lesion was significantly smaller, usually only pinhead sized.

The personal well-being on a scale of 1-10 rose in the median from 6.9 to 1.4.

The severity (assessed by the doctor) on a scale of 1-5 improved in the median from 3.8 to 1.4.

For 40% of the subjects, there was no reactivation phase of the herpes viruses after taking the two-phase preparation according to the present invention. These subjects were therefore free of symptoms up to the first outbreak during the observation period of six months. It has been suggested that this is a case of lacking immune competence due to consumer situations, analogous to the situation in competitive athletes (Type 1, see above). Here, only the intake of micronutrients was insufficient.

The consumption of the two-phase preparation according to the invention was able to improve the immune competence in the type 1 group to the extent that the persistent herpes simplex viruses in the body have been prevented from reactivation.

### Example 4

### Cell Experiments for the Vitality of Cells

These experiments were performed at the Heinz Nixdorf Chair for Medical Electronics at the Ludwig-Maximilians-University of Munich.

Antioxidants act as scavengers for free radicals and thus make important contributions to cell protection by destroying cell-damaging free radicals. The two-phase preparation according to the invention contains antioxidants.

To verify the effectiveness of the two-phase preparation according to the invention against attacks from free radicals, cell cultures were fed, in addition to their normal nutrition, with the two-phase preparation according to the invention, and then exposed to oxidative stress by hydrogen peroxide (H₂O₂). To compare the effectiveness two further groups were observed.

Group 1 consisted of cell cultures with normal nutrition, without the two-phase preparation according to the invention and oxidative stress. Group 2 had normal nutrition, with the two-phase preparation according to the invention and oxidative stress. Group 3 was the control group (i.e., with normal nutrition and without the two-phase preparation according to the invention and without oxidative stress).

As a measure of the vitality of the cells, the oxygen consumption of the cell cultures was used over time.

It was performed with a lab-on-a-chip from the firm HP-Med (HP-Medizintechnik GmbH; “Intelligent Micropate Reader”, IMR; http://www.hp-med.com/de/lab-on-a-chip-system-for-monitoring-of-living-cells.html, as of 16 Aug. 2011; Becker, B. et al., LaborPraxis March 2010). 30,000 Mouse-fibroblasts of the cell line L929 were sown per measurement chamber. Then it was pre-incubated for 22 hours, and then serum-free incubated for 80 minutes with an aqueous solution of the blue capsule according to Example 1B (1 capsule per 15 liters of water) and subsequently exposed to oxidative stress for 40 minutes by 0.05 mM H₂O₂. Thereafter, it was further normally incubated, and then measured in the standard medium for 24 hours on the analysis platform.

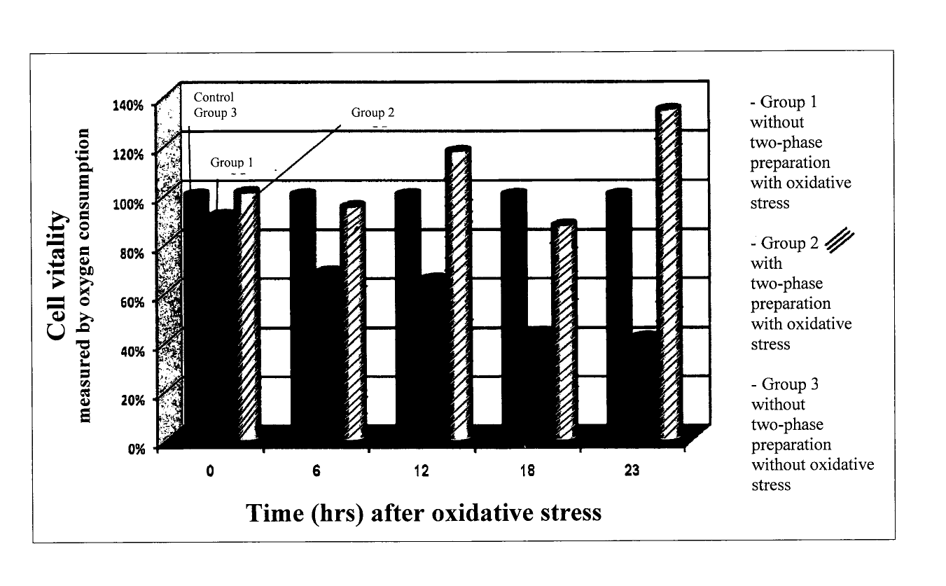

The results of the measurements are shown in FIG. 1. Compared to oxygen consumption of the cells of the control Group 3 [left column] (set to 100 percent), the oxygen consumption decreased in Group 1 (with oxidative stress, without the two-phase preparation according to the invention) [middle column] within the measurement period of 23 hours to about 40 percent. Subsequent investigation of the cells revealed that many cells of this culture were dead or severely damaged. In Group 2 [right column], which was additionally fed with the two-phase preparation according to the invention, however, the oxygen consumption increased to about 130 percent. Subsequent investigation of the cell culture revealed a healthy cell structure without dead or damaged cells. This shows that the two-phase preparation according to the invention may protect the cell structure from the otherwise harmful effect caused by oxygen radicals.

This result is to be interpreted that the two-phase preparation according to the invention may significantly improve the vitality of cells in vitro as well as in vivo. The invention is further described by the following numbered paragraphs:

1. Two-phase preparation for use in a chronologically staggered manner comprising
(a) a phase (A) comprising, in addition to water-soluble vitamins and/or the corresponding pro-vitamins, plant constituents and optionally minerals and/or trace elements, in a first unit dose form
(b) a phase (B) comprising, in addition to fat-soluble vitamins and/or the corresponding pro-vitamins, plant constituents and optionally minerals and/or trace elements, in a second unit dose form,
characterized in that phase (A) preferably contains grape seed extract and/or cranberry juice powder as plant constituents and that phase (B) preferably contains green tea extract and/or pomegranate extract as plant constituents.

2. Two-phase preparation according to paragraph 1, wherein
phase (A) comprises D-pantothenic acid (preferably as calcium D-pantothenate), niacin (preferably as nicotinamide), vitamin B₆ (preferably as pyridoxine HCl), riboflavin (vitamin B₂), vitamin B₁ (preferably as thiamine mononitrate), vitamin B₁₂ (preferably cyanocobalamin), and/or folic acid, as water-soluble vitamins

and/or
phase (B) comprises vitamin E, vitamin D₃, and/or beta-carotene, as fat-soluble vitamins.

3. Two-phase preparation according to paragraph 1 or 2, wherein
phase (A) comprises 40 mg-160 mg of grape seed extract, preferably 60 mg-120 mg of grape seed extract, and/or 20 mg-80 mg of cranberry juice powder, preferably 20 mg-80 mg of cranberry juice powder as plant constituents

and/or
phase (B) comprises 5 mg-160 mg of green tea extract, preferably 40 mg-120 mg of green tea extract, and/or 5 mg-80 mg of pomegranate extract, preferably 20 mg-60 mg of pomegranate extract as plant constituents.

4. Two-phase preparation according to any of the preceding paragraphs, wherein
phase (A) additionally comprises selenium as a trace element, preferably in the form of sodium selenite, and/or magnesium as a mineral, preferably in the form of a magnesium salt, such as magnesium citrate

and/or
phase (B) additionally comprises vitamin C, biotin, and/or zinc as a trace element, preferably in the form of a zinc salt, such as zinc gluconate.

5. Two-phase preparation according to any of the preceding paragraphs, wherein
phase (A) comprises 20 μg-90 μg selenite, preferably 45 μg-65 μg selenite, and/or 30 mg-90 mg magnesium, preferably 50 mg-70 mg magnesium

and/or
phase (B) comprises 120 mg-480 mg vitamin C, preferably 180 mg-300 mg vitamin C, and/or 20 μg-200 μg biotin, preferably 50 μg-150 μg biotin, and/or 1 mg-20 mg zinc.

6. Two-phase preparation according to any of the preceding paragraphs, wherein phase (A) comprises
x. 30 mg-90 mg magnesium, preferably in the form of magnesium citrate
xi. 40 mg-160 mg grape seed extract
xii. 20 mg-80 mg cranberry juice powder
xiii. 8 mg-28 mg of D-pantothenic acid, preferably in the form of calcium D-pantothenate
xiv. 6 mg-26 mg of niacin, preferably as nicotinamide
xv. 20 μg-90 μg of selenium, preferably as sodium selenite, 1% maltodextrin powder preparation
xvi. 1 mg-7 mg of vitamin B₆, preferably in the form of pyridoxine HCl
xvii. 1 mg-7 mg riboflavin (vitamin B₂)
xviii. 1 mg-7 mg vitamin B₁, preferably in the form of thiamine mononitrate
xix. 3 μg-12 μg vitamin B₁₂, preferably as cyanocobalamin as 1% inulin powder preparation
xx. 0.1 mg-1 mg folic acid.

7. Two-phase preparation according to any of the preceding paragraphs, wherein phase (B) comprises
i. 120 mg-480 mg of vitamin C
ii. 5 mg-160 mg of green tea extract
iii. 2 mg-6 mg of beta-carotene, preferably as 10% preparation
iv. 1 mg-20 mg of zinc, preferably as zinc gluconate
v. 5 mg-80 mg of pomegranate extract
vi. 5 mg-35 mg of vitamin E, preferably as dl-alpha-tocopheryl acetate
vii. 20 μg-200 μg biotin, preferably as 1% preparation
viii. 0.5 μg-10 μg of vitamin D₃, preferably as cholecalciferol with preferably 100.000 IU/g.

8. Two-phase preparation according to any of the preceding paragraphs, wherein phase (A) and/or phase (B) comprises further excipients, such as magnesium stearate, silicon dioxide, titanium dioxide, gelatin, and/or dyes, such as Indigo Carmin-Blue 2 or iron oxide.

9. Kit comprising the phases (A) and (B) according to any of the preceding paragraphs, wherein the unit dose forms of phases (A) and/or (B) are capsules, tablets, coated tablets, pills, granules, effervescent tablets, powders, drinkable solutions or suspensions.

10. Kit according to paragraph 9, wherein the dosage forms of phases (A) and (B) have different colors, and phase (A) is preferably colored with Indigo Carmin-Blue 2, and phase (B) is preferably colored with iron oxide orange or red, and both phases are preferably differently shaped capsules.

11. Use of a two-phase preparation according to any of paragraphs 1 to 8 or of a kit according to paragraph 9 or 10 as a food supplement and/or dietary substance.

12. Two-phase preparation according to any of paragraphs 1 to 8 or kit according to paragraph 9 or 10 for use as a medicament, preferably for use to support the immune system, particularly preferred for use in the treatment or prevention of viral diseases by supporting the immune system, in particular for use in the treatment or prevention of herpes.

13. Two-phase preparation according to any of paragraphs 1 to 8 or kit according to paragraph 9 or 10 for use in the treatment and/or prevention of recurring outbreaks of herpes simplex.

14. Use according to any of paragraphs 11 to 13, wherein phase (A) and phase (B) are alternatingly applied at a time interval of at least 2 hours, preferably of 2, 5, 8, 12, 24, or 28 hours, wherein the administration preferably starts with phase (A).

15. Use according to any of the paragraphs 11 to 14, wherein
(a) the phase (A) is applied to meals on days X+2N, where “N” represents the non-negative integers and
(b) the phase (B) is applied to meals on days X+(2N+1), where “n” represents the non-negative integers.

16. Use according to paragraph 15, wherein the meals are breakfast or dinner.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A two-phase preparation for use in a chronologically staggered manner comprising
(a) a phase (A) comprising, in addition to water-soluble vitamins and/or the corresponding pro-vitamins, plant constituents and optionally minerals and/or trace elements, in a first unit dose form
(b) a phase (B) comprising, in addition to fat-soluble vitamins and/or the corresponding pro-vitamins, plant constituents and optionally minerals and/or trace elements, in a second unit dose form,
characterized in that phase (A) preferably contains grape seed extract and/or cranberry juice powder as plant constituents and that phase (B) preferably contains green tea extract and/or pomegranate extract as plant constituents.

2. The two-phase preparation according to claim 1, wherein
phase (A) comprises D-pantothenic acid (preferably as calcium D-pantothenate), niacin (preferably as nicotinamide), vitamin B₆ (preferably as pyridoxine HCl), riboflavin (vitamin B₂), vitamin B₁ (preferably as thiamine mononitrate), vitamin B₁₂ (preferably cyanocobalamin), and/or folic acid, as water-soluble vitamins
and/or
phase (B) comprises vitamin E, vitamin D₃, and/or beta-carotene, as fat-soluble vitamins.

3. The two-phase preparation according to claim 1, wherein
phase (A) comprises 40 mg-160 mg of grape seed extract, preferably 60 mg-120 mg of grape seed extract, and/or 20 mg-80 mg of cranberry juice powder, preferably 20 mg-80 mg of cranberry juice powder as plant constituents
and/or
phase (B) comprises 5 mg-160 mg of green tea extract, preferably 40 mg-120 mg of green tea extract, and/or 5 mg-80 mg of pomegranate extract, preferably 20 mg-60 mg of pomegranate extract as plant constituents.

4. The two-phase preparation according to claim 1, wherein
phase (A) additionally comprises selenium as a trace element, preferably in the form of sodium selenite, and/or magnesium as a mineral, preferably in the form of a magnesium salt, such as magnesium citrate
and/or
phase (B) additionally comprises vitamin C, biotin, and/or zinc as a trace element, preferably in the form of a zinc salt, such as zinc gluconate.

5. The two-phase preparation according to claim 1, wherein
phase (A) comprises 20 μg-90 μg selenite, preferably 45 μg-65 μg selenite, and/or 30 mg-90 mg magnesium, preferably 50 mg-70 mg magnesium
and/or
phase (B) comprises 120 mg-480 mg vitamin C, preferably 180 mg-300 mg vitamin C, and/or 20 μg-200 μg biotin, preferably 50 μg-150 μg biotin, and/or 1 mg-20 mg zinc.

6. The two-phase preparation according to claim 1, wherein phase (A) comprises
x. 30 mg-90 mg magnesium, preferably in the form of magnesium citrate
xi. 40 mg-160 mg grape seed extract
xii. 20 mg-80 mg cranberry juice powder
xiii. 8 mg-28 mg of D-pantothenic acid, preferably in the form of calcium D-pantothenate
xiv. 6 mg-26 mg of niacin, preferably as nicotinamide
xv. 20 μg-90 μg of selenium, preferably as sodium selenite, 1% maltodextrin powder preparation
xvi. 1 mg-7 mg of vitamin B₆, preferably in the form of pyridoxine HCl
xvii. 1 mg-7 mg riboflavin (vitamin B₂)
xviii. 1 mg-7 mg vitamin B₁, preferably in the form of thiamine mononitrate
xix. 3-12 μg vitamin B₁₂, preferably as cyanocobalamin as 1% inulin powder preparation
xx. 0.1 mg-1 mg folic acid.

7. The two-phase preparation according to claim 1, wherein phase (B) comprises
i. 120 mg-480 mg of vitamin C
ii. 5 mg-160 mg of green tea extract
iii. 2 mg-6 mg of beta-carotene, preferably as 10% preparation
iv. 1 mg-20 mg of zinc, preferably as zinc gluconate
v. 5 mg-80 mg of pomegranate extract
vi. 5 mg-35 mg of vitamin E, preferably as dl-alpha-tocopheryl acetate
vii. 20 μg-200 μg biotin, preferably as 1% preparation
viii. 0.5 μg-10 μg of vitamin D₃, preferably as cholecalciferol with preferably 100.000 IU/g.

8. The two-phase preparation according to claim 1, wherein phase (A) and/or phase (B) comprises further excipients, such as magnesium stearate, silicon dioxide, titanium dioxide, gelatin, and/or dyes, such as Indigo Carmin-Blue 2 or iron oxide.

9. A kit comprising the phases (A) and (B) according to claim 1, wherein the unit dose forms of phases (A) and/or (B) are capsules, tablets, coated tablets, pills, granules, effervescent tablets, powders, drinkable solutions or suspensions.

10. The kit according to claim 9, wherein the dosage forms of phases (A) and (B) have different colors, and phase (A) is preferably colored with Indigo Carmin-Blue 2, and phase (B) is preferably colored with iron oxide orange or red, and both phases are preferably differently shaped capsules.

11. A food supplement and/or dietary substance comprising the two-phase preparation according to claim 1.

12. A method for supporting the immune system, particularly preferred for treating or preventing viral diseases by supporting the immune system, in particular for treating or preventing herpes comprising administering the two-phase preparation according to claim 1.

13. A method of treating and/or preventing of recurring outbreaks of herpes simplex comprising administering the two-phase preparation according to claim 1.

14. The method of claim 12, wherein phase (A) and phase (B) are alternatingly applied at a time interval of at least 2 hours, preferably of 2, 5, 8, 12, 24, or 28 hours, wherein the administration preferably starts with phase (A).

15. The method of claim 13, wherein phase (A) and phase (B) are alternatingly applied at a time interval of at least 2 hours, preferably of 2, 5, 8, 12, 24, or 28 hours, wherein the administration preferably starts with phase (A).

16. The food supplement and/or dietary substance of claim 11, wherein
(a) the phase (A) is applied to meals on days X+2N, where “N” represents the non-negative integers and
(b) the phase (B) is applied to meals on days X+(2N+1), where “n” represents the non-negative integers.

17. The food supplement and/or dietary substance of claim 16, wherein the meals are breakfast or dinner.

18. The method of claim 12, wherein
(a) the phase (A) is applied to meals on days X+2N, where “N” represents the non-negative integers and
(b) the phase (B) is applied to meals on days X+(2N+1), where “n” represents the non-negative integers.

19. The method of claim 18, wherein the meals are breakfast or dinner.

20. The method of claim 13, wherein
(a) the phase (A) is applied to meals on days X+2N, where “N” represents the non-negative integers and
(b) the phase (B) is applied to meals on days X+(2N+1), where “n” represents the non-negative integers.

21. The method of claim 20, wherein the meals are breakfast or dinner.
